(19) United States
(12) Patent Application Publication
(10) Pub. No.: US 2024/0233901 A1
(43) Pub. Date: Jul. 11, 2024

(54) PORTABLE MEDICATION/DRUG IDENTIFICATION APPARATUS WITH DOCUMENTATION, LABELING AND METHOD OF DRUG MANAGEMENT

(71) Applicant: Codonics, Inc., Middleburg Heights, OH (US)

(72) Inventors: Gary W. ENOS, Hudson, OH (US); Thomas G. SPAPE, Twinsburg, OH (US); Lawrence SRNKA, Northfield Center, OH (US)

(21) Appl. No.: 18/239,991

(22) Filed: Aug. 30, 2023

Related U.S. Application Data
(60) Provisional application No. 63/438,140, filed on Jan. 10, 2023.

(51) Int. Cl. G16H 20/13; A61G 3/00; A61J 7/00; G16H 10/60; G16H 40/67; G16H 70/40

(52) U.S. Cl. CPC G16H 20/13; A61G 3/001; A61J 7/0084; G16H 10/60; G16H 40/67; G16H 70/40; G06K 7/1413

(57) ABSTRACT

Provided are computing system and method for documenting and communication information within a mobile medical care environment. A mounting system supports the computing system within a mobile medical care environment. The computing system includes a decoding module that causes the processing circuitry to scan, with the non-contact scanner, a computer-readable code encoding information about a first medicinal substance. A record module causes the processing circuitry to link at least a portion of the encoded information from the computer-readable code to a patient ID that is associated with the patient and stored in the memory. A communication module transmits (i) the portion of the information from the barcode linked to the patient ID, and (ii) the patient ID to be stored by a medical record system at a healthcare destination.

### BACKGROUND OF THE INVENTION

### 1. Field of the Invention

This application relates generally to a drug identification, documentation, and labeling apparatus and method for confirming & recording drug preparation and communicating records of such usage. More particularly, the present application relates to a portable apparatus and method for documenting drugs and recording/communicating drug administration remotely, producing a drug label with a machine-readable code in a mobile or remote medical environment, and electronically communicating information about usage of the drug to a remote destination.

### 2. Description of Related Art

Conventional drug vial selection and consumption are error prone and lack real-time documentation and logging of use. Manual recording and labeling suffer from many drawbacks, and have limited reliability due primarily to “after the fact” recording from memory, failure to report, and other human errors. Sloppy handwriting can make the record and label difficult to read, or altogether illegible. Each technician who selects and prepares medications may also do so in a different manner, or record different content to record than another technician. In such situations, the selection record and documentation of what, when, where, why and by whom a drug is consumed is left open to interpretation, and often lacks information essential for proper documentation and record keeping purposes.

Further, manual record keeping and drug labeling methods generally lack the ability to generate “in real time” labels & records that are useful to facilitate a secure double check that the proper patient is to receive a drug to be administered via an automated drug delivery device. Traditionally, human-readable vial labels and hand-written prepared drug label content have been manually compared to other human-readable content identifying the patient at the location where the drug is to be administered to the patient.

More recently, attempts have been made to at least partially automate the drug identification and recorded use labeling process, relying on computer-readable codes to obtain a portion of the information required to generate the prepared drug recode and drug label. Such systems rely on timely updates to databases within a hospital setting where such fixed labeling installations are placed to ensure drug information is up to date. Further, such fixed installations can be connected to a hospital network to receive and transmit data concerning drug label preparations. However, drug label preparation is also required outside of the hospital setting or at various different locations within a hospital, away from where such fixed installations are located. Furthermore, drugs prepared remotely within a hospital or other health procedure environments often lack connectivity for real-time communication of recorded selection and use.

### BRIEF SUMMARY OF THE INVENTION

In the United States, considering emergency services for example, there are approximately 27,000 fire departments listed with the National Fire Department Registry. The 2020 National EMS Assessment reported a total of 1,030,760 licensed EMS professionals, from emergency medical responders to paramedics, in the United States, affiliated with over 19,000 Credentialed EMS Agencies, operating over 70,000 Credentialed EMS Vehicles. Such resources are used to respond to nearly 28.5 million 911 dispatches every year in 41 states.

The potential for improper medication delivery to patients extends to the remote & mobile healthcare facilities and modes of transportation utilized in responding to emergency calls and patient transports, for example. Accountability of used and wasted drugs during patient treatment is not only a hospital pharmacy or clinical environmental problem. Medication selection, preparation, and use documentation are concerns at all locations where patient care is performed. Pharmaceutical audits and control require increased scrutiny including remote medication access and use locations. It is the same DOP Pharmacists that control inside and outside world of drug delivery and accountability. Stocking of drugs including antidotes and documented basis for re-stock applies to mobile healthcare environments as well. Ambulance controlled substance accountability is an area DOP needs Realtime/online system communicated information (per DEA number assigned to the service). Integration with LIFEPAK 15 emergency care and data connectivity, as well as other systems can be implemented as described herein to manage patient care information to easily and securely collect and send patient information is needed. Procedural area and patient care areas need digital confirmation and communication of drug access and use to clinical information systems, and documentation of drug and diluent use tied to patient ID are currently performed manually, after-the-fact once patients arrive at their healthcare-related destination.

National Pre-Hospital and Hospital Data Integration is an aspect of the present disclosure. The National EMS Information System (NEMSIS) is a national effort to standardize the type of data collected by EMS agencies. NEMSIS provides the framework for collecting, storing, and sharing standardized EMS data from states nationwide. Furthermore, EMS Data-National Highway Traffic Safety Administration (NHTSA) is a key focus involved with the health data initiatives.

To solve at least a portion of potential problems associated with the conventional art, the present application involves portable, hand-held and/or fixture mounted “smart scanner-computer” (a “terminal”) with an ability to maintain current “approved” UDI/NDC drug database and “on-hand” stock-levels. The terminal allows for fast scanning of drug with audible/visual confirmation of drug in hand based on a computer-readable code such as the National Drug Code (“NDC”).

A user interface such as a LCD or LED display can be utilized as a touch screen or in conjunction with a keyboard or other input device to enable an operator to enter patient reference (name, ID, JDoe-1, etc) to document emergency and scheduled administration used and time used. For example, a paramedic/EMS Life Officer can be required to logon by entering login credentials to access drugs and document their usage within the mobile environment. A device with WiFi/Bluetooth connectivity for communication with CIS/LIFEPAK, and other healthcare informatics to document selection, use, and disposal of drugs or other substances is within the scope of the present disclosure.

According to other embodiments, an onboard display and/or speaker provided to a mobile healthcare environment such as an ambulance can issue an alert to confirm drug selection, with the drug being identified in response to scanning a computer-readable code such as a barcode, RFID tag, etc. The identified drug information can be used to track available stock by decrementing the quantity within the mobile environment, and increasing the quantity in response to scanning a computer-readable code as part of a restocking process. The terminal can optionally upload records to be processed or maintained at a hospital or other healthcare facility, or a dispatch location for example, through a cellular communication link, upon entering the range of a wireless network upon arriving at the facility, through a secure communication standard between the mobile environment and the facility, or in any other manner. The terminal can optionally communicate with a printer (if available), to print a label for a drug, print usage/consumption information, etc.

Although the terminal is described herein as being installed within an ambulance as an example of a mobile environment, the present disclosure is not so limited. Alternate embodiments include a handheld device, a device installed within an ambulance or other transport vehicle such as a medical helicopter, or anywhere drugs are prepared (nursing stations, patient floors, procedural centers/areas, ambulance/EMS Squad, ER departments, Urgent Care environments, Skilled Nursing/Long Term Care facilities, etc.

According to some embodiments, a computing system is disposed within a mobile medical care environment such as an ambulance, for example, that comprises a drug dispensary storing a supply of medicinal substances available for use in administering care to a patient within the mobile medical care environment. The computing system includes: a mounting system that supports the computing system within the mobile medical care environment; processing circuitry; a non-contact scanner; and a memory coupled to the processing circuitry. The memory stores a decoding module including instructions that, when executed by the processing circuitry cause the processing circuitry to scan, with the non-contact scanner, a computer-readable code encoding information about a first medicinal substance stored by the drug dispensary. The memory can store a record module including instructions that, when executed by the processing circuitry cause the processing circuitry to link at least a portion of the encoded information to a patient ID that is associated with the patient and stored in the memory. The memory can store a communication module including instructions that, when executed by the processing circuitry cause the processing circuitry to transmit: (i) the portion of the encoded information linked to the patient ID, and (ii) the patient ID to be stored by a medical record system at a destination medical care environment.

According to some embodiments, the present technology comprises a method of documenting a drug within a mobile medical care environment that comprises a drug dispensary storing a supply of medicinal substances available for use in administering care to a patient within the mobile medical care environment. The method includes, with a computing system mounted within the mobile medical care environment: scanning a computer-readable code encoding information about a first medicinal substance stored by the drug dispensary with a non-contact scanner; linking at least a portion of the encoded information to a patient ID that is associated with the patient in the mobile medical care environment and stored in the memory; and transmitting via an antenna provided to the mobile medical care environment: (i) the portion of the encoded information linked to the patient ID, and (ii) the patient ID to be stored by a medical record system at a destination medical care environment.

The above summary presents a simplified summary of the problem and solution in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF SEVERAL VIEWS OF THE DRAWING

The invention may take physical form in certain parts and arrangement of parts, embodiments of which will be described in detail in this specification and illustrated in the accompanying drawings which form a part hereof and wherein:

FIG. 1 shows an illustrative embodiment of a labeling apparatus for generating labels to be applied to medicinal substances in a medical facility;

FIG. 2 shows a block diagram schematically depicting components of a labeling apparatus for generating labels to be applied to medicinal substances in a medical facility;

FIG. 3 shows an illustrative embodiment of a medical labeling network arrangement at a medical facility;

FIG. 4 shows an illustrative embodiment of a vehicle-mounted computer terminal for identifying and documenting drugs within a mobile environment;

FIG. 5 shows an illustrative embodiment of a first handheld computer terminal for identifying and documenting drugs within a mobile environment;

FIG. 6 shows an illustrative embodiment of a label printed in accordance with the present disclosure;

FIG. 7 shows an illustrative embodiment of a mobile healthcare environment in the form of an ambulance;

FIG. 8 shows an interior view of an illustrative embodiment of a patient module of the ambulance shown in FIG. 8; and

FIG. 9 shows a flow diagram graphically depicting an illustrative method of documenting the use and/or administration of a drug in a mobile healthcare environment.

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

It is also to be noted that the phrase “at least one of”, if used herein, followed by a plurality of members herein means one of the members, or a combination of more than one of the members. For example, the phrase “at least one of a first widget and a second widget” means in the present application: the first widget, the second widget, or the first widget and the second widget. Likewise, “at least one of a first widget, a second widget and a third widget” means in the present application: the first widget, the second widget, the third widget, the first widget and the second widget, the first widget and the third widget, the second widget and the third widget, or the first widget and the second widget and the third widget.

The present disclosure involves a method and apparatus for documenting at least one of: drug administration and drug consumption/replenishment in a mobile healthcare environment such as an ambulance, an embodiment of which is shown in FIG. 7. According to the embodiment shown in FIG. 7, the ambulance 1 is a wheeled vehicle that is licensed to navigate public roadways to transport patients to brick-and-mortar healthcare destinations such as hospitals and urgent care centers. The ambulance 1 includes a cab 2 in which a driver operates the ambulance 1, and a patient module 4 in which a patient can receive medical attention while being transported to the healthcare destination.

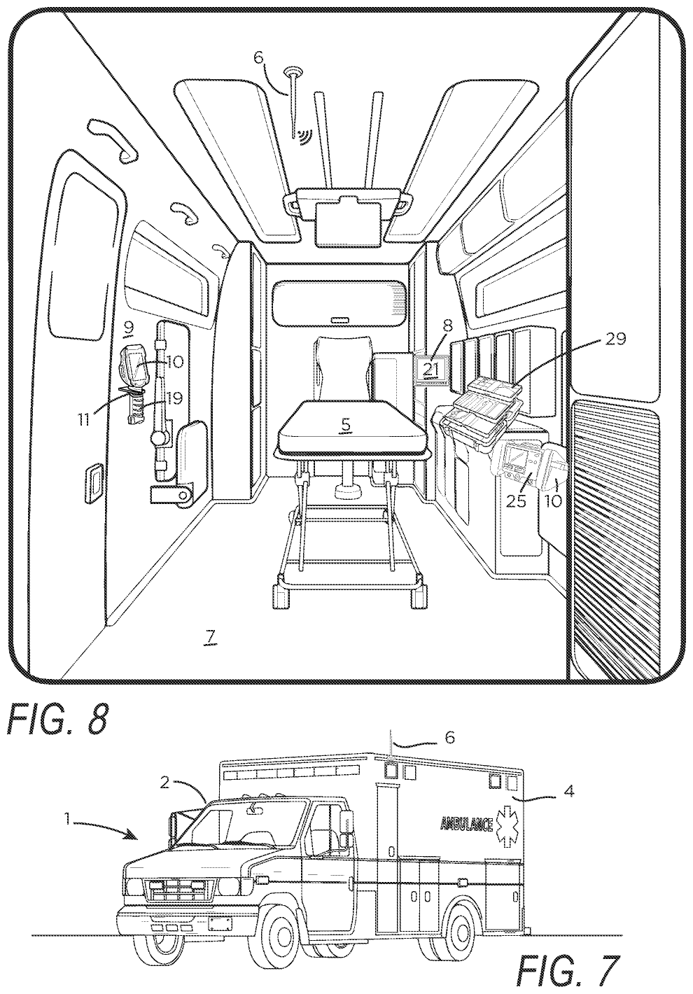

FIG. 8 shows an interior of an illustrative embodiment of the patient module 4 of the ambulance 1. The illustrated patient module 4 contains a gurney 5 that is secured to a floor 7 of the patient module 4 to support the patient during transport. The patient can be strapped to the gurney 5, which can be engaged by wheel locks affixed to the floor 7, thereby securing the patient in place during transport.

The ambulance 1 can also be equipped with a communication system that conducts two-way communications between the ambulance 1 and at least a remote location such as the healthcare destination to which the ambulance 1 is en route, a dispatch center associated with the ambulance 1 (e.g., a facility operated by a corporate entity that operates the ambulance 1), or another remote location. The remote location is considered “remote” in that the ambulance 1 is not on the real property where the destination healthcare facility is located. According to some embodiments, the communication system includes at least an antenna 6 that transmits and receives wireless signals transmitted between the remote location and the ambulance 1, and a communication terminal 8 that includes electronic circuitry to modulate input data introduced to the communication terminal 8 into the signals transmitted via the antenna 6 to the remote location. For example, voice and/or alphanumeric text can be input to the communication terminal 8, modulated by the communication terminal 8 into an output signal, and transmitted by the antenna 6 to a compatible communication terminal at, or associated with the destination healthcare facility, a dispatch center or other recipient. The antenna 6 can optionally be disposed within the patient module 4 or another compartment of the ambulance 1, or positioned externally of the patient module 4 as shown in FIG. 7. The communication terminal 8 can include a citizens band radio or any other mobile radio system that communicates via a broadcast, cellular or satellite communication system. The communication terminal 8 can optionally be paired or otherwise linked to the portable communication device 54, for example. A display device 21 can also optionally be provided to the communication terminal 8 to visually present information and/or receive input pertaining to the medical care of a patient being transported.

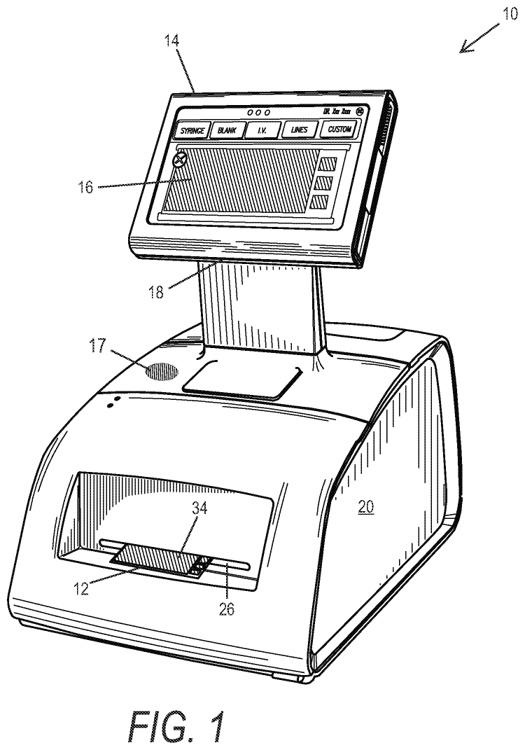

An illustrative embodiment of a computer terminal 10 that can be installed in the patient module 4 or on a wheeled cart in a hospital for example, is shown in FIG. 1. The computer terminal 10 includes a touch-screen display 14 that can be pivotally coupled to a cabinet 20 to display a virtual label 16 comprising label content 34 that will be printed onto a label 12 that will be applied to a medicinal substance. The computer terminal 10 can be operable to scan a computer-readable code and print a label to be applied to a medical container such as a syringe as described in U.S. patent application Ser. No. 12/901,110, which is incorporated by reference herein in its entirety. The display 14 can display soft keys that, when touched by a technician or any other user, inputs data and commands into the computer terminal 10. The virtual label 16 is a computer-generated rendering of the label 12 that offers the user visual confirmation of the appearance of the physical label 12 to be printed by a printer 26. A computer-input peripheral such as a non-contact scanner 18, for example, can be provided at a convenient location, such as integrally formed in a bottom portion of the display 14 to read a machine-readable code supported beneath the scanner 18 for example. Integrally forming the scanner 18 as part of the display 14 provides for space savings over an arrangement where the scanner 18 is formed as a separate peripheral, which can be repositioned relative to the display 14. However, other embodiments can allow for a separate and distinct scanner 18 and/or display 14.

The computer-input peripheral can be a barcode reader or radio-frequency identification (“RFID”) tag reader, or any other device that reads a machine-readable code such as a barcode or RFID code, respectively, or any other machine-readable code without requiring contact between the computer terminal and the code, and optionally the user during entry of the code. According to alternate embodiments, the display 14 can be utilized by a user as the computer-input peripheral. For such embodiments, the soft keys displayed by the display 14 can be selected to input information such as a medicinal substance being prepared to be administered to a patient or other information to be utilized in generating the label as described herein. According to yet alternate embodiments, a speaker 17 can optionally be provided to the display 14 or any other portion of the computer terminal 10 to broadcast audible sounds.

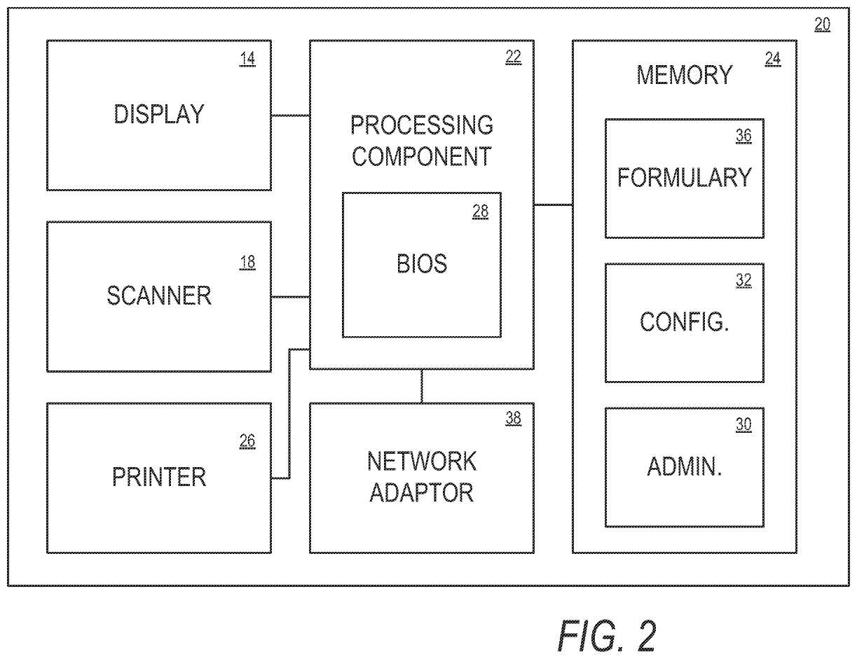

The computer terminal 10 also includes a cabinet 20 that houses or supports components that are operable to produce the label 12 in compliance with a medical labeling standard. But if what is being labeled is anything other than the medicinal substance, then the label 12 produced is to be compliant with a standard developed by a trade or professional organization, governing body, government agency, a healthcare provider or facility such as a hospital, or any other standards body setting forth policies for labeling such material. The internal components housed within the cabinet 20 are schematically illustrated by the block diagram of FIG. 2. The components can be formed from an arrangement of computer hardware such as ASICs, computer processors, programmable logic controllers and other circuitry; or a combination of computer hardware and computer-executable instructions.

For example, a processing component 22 in the form of a microprocessor-based circuitry is provided to execute computer-executable instructions stored in a non-transitory, computer-readable memory 24 such as a hard disk drive, read-only memory (“ROM”), random access memory (“RAM”), optical disc, or any other suitable memory device, or any combination thereof. The computer-executed instructions, when executed by the processing component 22, result in the performance of the method of generating a label for a medicinal substance described in detail below. A BIOS 28 is provided to load the operating system and other such administrative instructions 30 stored in the memory 24 and manage hardware interface permissions of the computer terminal 10. The operating system can be configured to only load authorized updates to prevent unauthorized changes to the formulary 36, configuration data 32 and administration instructions 30. Configuration data 32 controls various features of the computer terminal 10 that are active and available for use at any given time. The configuration data 32 can optionally be stored, updated and deleted from the memory 24 by the introduction of a so-called smart drive comprising a USB compatible flash memory to the computer terminal 10. When the smart drive is introduced to the computer terminal 10, it establishes the configuration data 32 of the computer terminal 10. The configuration data 32 can optionally be used to deactivate functional features that the computer terminal 10 would otherwise be able to perform based on the model of the computer terminal 10 purchased. Accordingly, a common hardware platform of the computer terminal 10 can be configured in a plurality of different functional configurations based on the configuration data 32.

In addition to the administrative instructions 30, the memory 24 also stores an updatable formulary 36 containing a database of medicinal substances that can be identified by the computer terminal 10 and select information for each medicinal-substance entry in the database. The formulary 36 of FIG. 2 can also optionally comprise computer-executable instructions that, when executed, establish a decoding module. The instructions of the decoding module, when executed by the processing component 22, cause the processing component to scan, with the non-contact scanner 18, a computer-readable code encoding information about a first medicinal substance stored by a drug cabinet 29, described below.

According to some embodiments, the formulary 26 can comprise a record module including instructions that, when executed by the processing component 22, cause the processing component 22 to link at least a portion of the encoded information to a patient ID that is associated with the patient to be transported in the ambulance 1 as described herein, and stored in the memory 24. A communication module of the formulary 36 can also include instructions that, when executed by the processing component 22, cause the processing component 22 to transmit: (i) the portion of the encoded information linked to the patient ID, and (ii) the patient ID to be stored by a medical record system at a destination medical care environment as described herein.

The database of medicinal substances stored by the formulary 36 can optionally be stored, updated and deleted from the memory 24 by the introduction of a so-called smart drive comprising a USB compatible flash memory to the computer terminal 10. When the smart drive is introduced to the computer terminal 10, it establishes the formulary 36 of the computer terminal 10. Illustrative examples of the select information that can be provided for the medicinal-substance entries includes, but is not limited to, an ID number such as a NDC code, UPC code, EAN code, or any other identifying data that can be used to relate a barcode or other computer-readable code to the medicinal-substance entries; a sound file that, when played, audibly announces the name of the medicinal substance identified in response to scanning a machine readable code; warning data; or any combination thereof.

According to some embodiments, the decoding module stored by the memory 24 comprises instructions that, when executed by the processing component 22, cause the processing component 22 to determine the portion of the encoded information based on a signal transmitted by the non-contact scanner 18 in response to scanning the computer-readable code applied to a drug vial. For example, the decoding module can look up an entry corresponding to content transmitted by the signal in the database of a formulary locally stored in non-transitory computer-readable medium onboard the ambulance 1. An example of the portion of the encoded information include at least one of a name of the medicinal substance, and is determined from the formulary locally within the ambulance 1 by the computer terminal 10, optionally without communicating with a remote computing terminal located externally of the ambulance 1 (e.g., at a healthcare destination). The communication module can include an interface with a communication system provided to the ambulance 1, to transmit the portion of the encoded information to a medical record system over the communication system of the ambulance 1 in real time, or upon entering the communication range of a local area network located at the healthcare destination, for example.

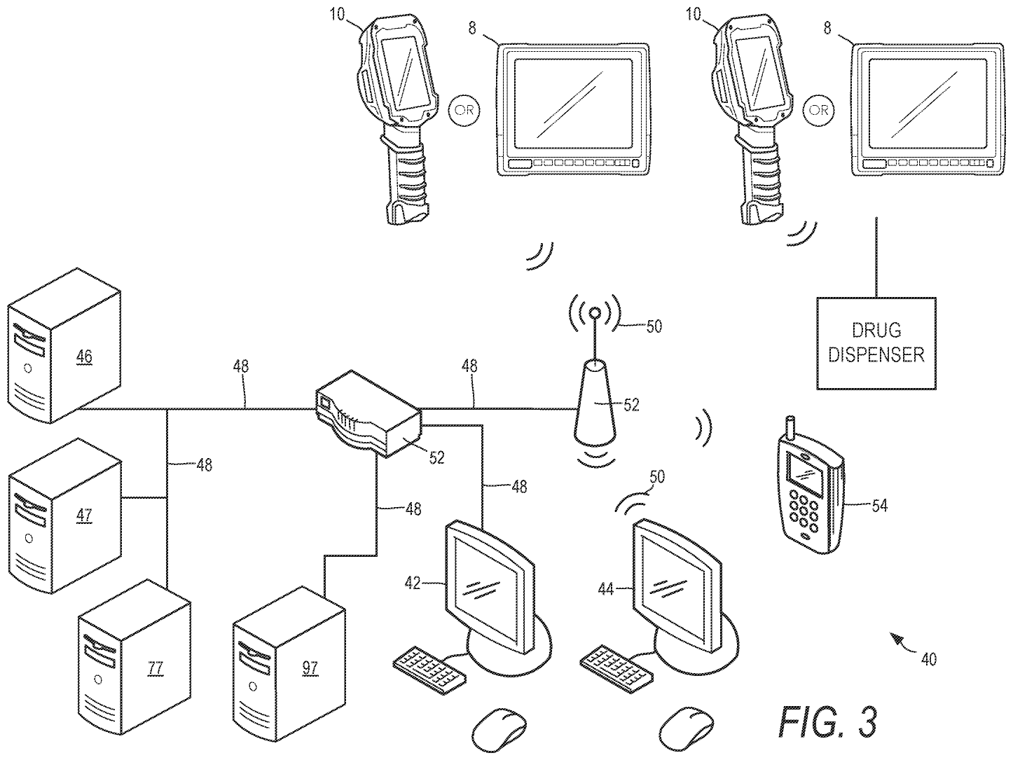

A network adaptor 38 is operatively connected to communicate with the processing component 22 for translating signals received by the computer terminal 10 over a network 40 at a medical facility, such as that illustrated in FIG. 3. The network adaptor 38 can be compatible with any type of network communication. For example, the network adaptor 38 can include a hardwired, 10Base-T, 100Base-T, or 1000Base-T Ethernet interface with an RJ-45 socket, a coaxial cable interface, a fiber-optic interface, any format of wireless communication interface such as an antenna compatible with any of the 802.11 standards established by the IEEE, or any combination thereof. Embodiments including wireless network adaptors 38 can employ any desired securing protocol such as WEP, WPA and WPA2, for example, and other suitable security protocol. For embodiments including a network adaptor 38 compatible to communicate over a plurality of different network communication channels, both a hard-wired communication portion of the network adaptor 38 and a wireless communication portion of the network adaptor 38 can optionally be concurrently active. Thus, the computer terminal 10 can optionally communicate via both the hard-wired and wireless portions of the network adaptor 38 concurrently.

As shown in FIG. 3, a plurality of the computer terminals, each referred to generally at 10 can be included in a network 40 arranged at a healthcare facility, within a fleet of ambulances 1 or other transport vehicles, or a combination thereof. For example, the patient module 4 of a plurality of ambulances 1 or other transport vehicles, a plurality of operating rooms in which surgical procedures take place at the healthcare destination, etc. may each contain one of the computer terminals 10 located therein. Other networks may include a computer terminal 10 in an examination room where procedures such as minimally invasive examinations of patients are conducted. Network connecting the computer terminals 10 can allow for data to be transmitted, directly or indirectly, to the healthcare destination while the ambulance 1 is en route, or after the ambulance 1 arrives at the healthcare destination. For example, usage data (e.g., a list of drugs for which labels have been prepared, information such as concentration and date/time included on a label 12 prepared for a drug in the mobile healthcare environment, etc.) and/or patient data (e.g., the patient ID for a person being transported in the ambulance 1 and for whom a drug is prepared and/or administered, etc.) can be transmitted.

The network 40 can also optionally include a pharmacy computer terminal 42 executing computer-executable instructions (referred to hereinafter as an administration tool or “AT”) that, when executed, manage one or more, and optionally all of the computer terminals 10. Each computer terminal 10 to be managed by the AT can be optionally assigned a user-specified designation using the AT to distinguish the computer terminals from each other on the network 40, and to optionally provide the user with a brief description of each computer terminal 10. For example, a computer terminal 10 located in operating room #1 can be assigned the designation OR-1 to indicate its location. According to alternate embodiments, the user-specified name Cart-1 (or drug bag or drug box/container or storage location if mobile) could be assigned to a computer terminal on mobile cart #1. As another example, the computer terminal 10 in the ambulance 1 can be assigned the user-specified name EMT1. An IT computer terminal 44 can also optionally be connected as part of the network 40 to execute the AT and allow technical personnel to manage technical aspects of the computer terminals 10, but optionally exclude from the permissions granted to technical personnel the ability to alter drug or other medical-related content stored by the computer terminals 10. The permissions granted to a user at the terminals 42, 44 can optionally be determined when the user logs in based on a username/password combination, a computer-readable identification, or any other identifying information. Thus, the terminals 42, 44 do not necessarily have to be dedicated solely for any particular purpose.

The pharmacy terminal 42 can be located in a pharmacy at a healthcare facility, at a dispatch center associated with the ambulance 1, or any other location where an inventory of controlled drugs and medicinal substances (hereinafter generally referred to as “drugs”) is maintained and used to replenish a drug cabinet 29 (FIG. 8), interchangeably referred to herein as a drug dispensary, within the patient module 4. The drug cabinet 29 stores a supply of medicinal substances available for use in administering care to a patient within the ambulance 1. A pharmacist or a plurality of pharmacists maintain and administer a master drug database (“MDD”) containing an identity, identification code (e.g., NDC) number, concentration and other pertinent information for drugs used by the pharmacy. Drugs are entered into the MDD by the pharmacist, and the terminals 42, 44, and optionally other terminals connected to the network 40 can restrict access to the MDD and prevent unauthorized individuals from entering or altering drug entries in the MDD, and optionally from accessing the MDD altogether. In other words, the pharmacist(s) registered and authorized to work at the health care facility and those they grant permission to access the MDD are the only individuals permitted to manipulate data in the MDD.

From the MDD, the pharmacist manages a formulary to be stored in the memory 24 of one or more of the computer terminals 10 using the AT with the pharmacist permission. For example, the formulary of the computer terminal 10 disposed within the patient module 4 of the ambulance 1 can be updated by pushing (e.g., transmitting over a wireless network 40) a new formulary from the AT, that replaces the existing formulary stored in the memory 24 in its entirety. The new formulary can be pushed to the computer terminal 10 in the patient module 4 if the communication system 8 is operatively connected to communicate over a wide area network such as a cellular phone network, a satellite communication network, or a terrestrial communication network. According to alternate embodiments, the updated formulary or other data to be transmitted to the computer terminal 10 in the patient module 4 can optionally be updated only while the antenna 6 of the ambulance is within range of a secure, local area network (e.g., a WiFi network compliant with an IEEE 802.1x standard) with a communication range substantially limited primarily to the real property of the destination healthcare facility, dispatch station, etc. The local area network antenna can optionally include a communication range that is insufficient to communicate with the medical record system at the healthcare destination while the ambulance 1 is traveling along a portion of a route to the healthcare destination, but sufficient to communicate with the medical record system when the ambulance 1 is located at (e.g., on the property of) the healthcare destination.

The formulary can include a subset of the MDD, and the subset can optionally comprise drugs that are commonly used in the ambulance 1 or other mobile healthcare environments at the healthcare facility where the computer terminal 10 is positioned. The same formulary can optionally be stored in the memory 24 of more than one computer terminal, and can optionally be customized to include drugs utilized during surgical procedures relating to a particular medical discipline. For example, the same formulary comprising drugs commonly used during cardiac surgical procedures may be stored in the memory 24 of computer terminals 10a, 10b, which are each located in a respective operating room dedicated for such procedures. Another, different formulary comprising drugs, optionally in appropriate doses, suitable to be administered to children can be stored in the memory 24 of a computer terminal 10 located in an operating room dedicated for pediatric surgical procedures. According to alternate embodiments, the formulary 36 stored in the memory 24 of a computer terminal 10 can be evaluated and updated, replaced or otherwise changed before each surgical procedure if the operating room where the computer terminal 10 is located is not dedicated for a particular type of surgical procedure. When a formulary update is needed to accommodate a specific type of procedure, a pharmacist's access can be required to update, replace or otherwise change the formulary in the computer terminals 10, and updating, replacing and changing the formulary in the memory 24 in each of the computer terminals 10 can be performed over the network as described in detail below.

In addition to a pharmacist's level of permission, there can be other permission levels limiting access to the computer terminals 10 to different users. For example, an anesthesiologist may be granted permission to use a computer terminal 10 to interrogate a barcode or other machine-readable code on a drug vial to extract the identity of the drug and print a label to be applied onto a syringe. The anesthesiologist can optionally also be granted permission to confirm that the interrogation of a barcode has returned the proper drug identification. However, the anesthesiologist may be prevented from editing the formulary stored in the memory 24 of the computer terminal 10.

Additionally, an IT professional can be granted permission to address any technical, computer hardware-and-software-related issues with the computer terminals 10 that are unrelated to the specific drug information of the MDD and/or formulary. For example, the IT professional may be granted permission to assign and/or change: an IP address of the computer terminals 10, a security protocol employed, and other computer-specific matters. However, some information related to the formulary such as the version and description of the formulary can be viewed by the IT professional to ensure that the proper computer terminal 10 has the correct formulary installation. This also applies to version and description information of the operating system, BIOS 28, configuration data 32 and administration instructions 30.

The network 40 in FIG. 3 also includes an email server 46 through which email is to be transmitted to individuals who perform tasks related to the computer terminals 10 at the healthcare facility. The email server 46, like the computer terminals 10, and optionally other resources of the network 40, can transmit signals to other network resources via hard-wired communication channels (represented by solid lines 48 in FIG. 3) such as CAT-5 Ethernet cable, via wireless communication channels (represented by arched, radiating signals 50), or a combination thereof. For example, email messages notifying individuals that a triggering event has occurred on one or more computer terminals 10 are transmitted from the email server 46 to one or more of the terminals 42, 44, a portable communication device 54 such as a personal digital assistant, cellular telephone, tablet or laptop computer, and the like. Additionally, the email server 46 can be configured to apply one or more rules that organize and deliver the information in more meaningful ways to the user. For example, a pharmacist may want notification of all problems with the formulary 36 (e.g., a “drug not found” notification) to be aggregated together and delivered to him at the start of his work shift and again 4 hours later. The email server 46 can be configured to transmit such notices in a single communication to the pharmacist at those times. Further, different pharmacists may prefer different notification procedures and different times at which such notifications are to be received, and the email server 46 can optionally be configured to satisfy the requests of each pharmacist individually. However, a group of IT technicians may want prompt notification of technical problems that prevent a computer terminal 10 from operating properly in a surgical suite. Again, the email server 46 can be configured to promptly transmit such notifications to the IT technicians substantially immediately upon detecting such technical problems.

Network resource allocation equipment 52 such as switches, routers, wireless access points, and the like can be included in the network 40 to share network resources and establish communication between the computer terminals 10 and the terminals 42, 44. Additionally, the computer terminals 10 can optionally serve as an expansion port to which other network resources such as the automated drug dispensing system 56, commonly referred to as a “smart cart”, can be connected to the network to dispense and document the strength, quantity and type of drug according to a schedule or in response to the occurrence of a predetermined event. Additionally, since one of the functions of smart carts is to control the dispensing of drugs and one of the functions of computer terminal 10 is producing labels for containers such as syringes that are filled with drugs from the smart cart, there are benefits related to efficiency if the devices can share information. For example, a network connection between the smart cart and computer terminal 10 will allow user login information such as username and password entered on one device to be shared with the other device so a user is authenticated on both devices with a single login. Other benefits include being able to share information about drugs being used in a procedure between the devices so verification and reconciliation of drugs can be performed to ensure the proper medications are dispensed, labeled and tracked for improving the accuracy of patient records and accurate billing. As shown in FIG. 3, the automated drug dispensing system 56 is hard-wired to the computer terminal 10c, which is connected wirelessly to other network resources.

Before the computer terminals 10 are usable in a mobile medical environment, the AT software can be installed on one or more of the terminals 42, 44 to be used by a pharmacist at a hospital or other health care facility to populate the MDD. The AT accepts drug information from various sources such as commercially available drug databases (e.g. Lexicomp) and allows the pharmacist to selectively add drugs to the MDD, which can be stored at network-accessible storage server or locally by the terminal 42, 44 running the AT. In simplest terms, the MDD is the set of drugs available to the hospital or other healthcare facility.

Once the MDD is populated with drug information, the pharmacist will use the AT to select a subset of drugs from the MDD to be added to the formulary that will be stored in the memory of one or more of the computer terminals 10, thereby enabling the computer terminals 10 to recognize the drugs in that formulary. The formulary managed using the AT running on one of the terminals 42, 44, as it pertains to the computer terminals 10, can be considered an official set of medications with associated information for preparing and labeling drug containers in accordance with a medical labeling standard. The “associated information” can include information for preparing the drug, which usually means diluting the drug when needed. It can also include information related to the color, patterns, graphics and textual information printed on the label for specific drugs to render those labels, once printed, compliant with the medical labeling standard. Other types of associated information can be files, data for implementing a computer-generated voice, references to files for audibly pronouncing the name of the drug and important drug related information such as the concentration value and concentration units, or any combination thereof. For example, in the case of Propofol 10 mg/ml, a single audio file, or more than one audio file or references to audio files can be combined together to audibly speak the drug name and concentration of the drug as “Propofol ten milligrams per milliliter”. According to the present example, the drug name “Propofol” can be contained in one audible file while the concentration value “ten” is in another audible file and the concentration units “milligrams per milliliter” in a third audible file. These three audio files can be executed and played in sequence to allow the computer terminal to audibly broadcast “Propofol ten milligrams per milliliter” via the speaker 17 in response to the scanning of a barcode associated with the container that contains 10 mg/ml Propofol. Other audible information including information about errors such as “do not use drug”, for example, can also be associated with a drug in the formulary using the AT. The “do not use drug” audible information can optionally be audibly output using the speaker 17 when a drug has been recalled and a pharmacist wants the computer terminals 10 to notify users not to use a drug that has been recalled, or is otherwise not suitable for use, for example. The computer terminal 10 can automatically assign some audible drug information by examination of the data related to the drug. For example, the concentration value 10 can be used to select the audible file or file reference that speaks the word “ten”. The same applies to the concentration units. mg/ml can automatically be used to select the audible file or file reference corresponding to “milligrams per milliliter”. Since the MDD can include information on many types of drugs used in the hospital including pills, capsules, ointments, patches, injectables, etc., the pharmacist can optionally select only the drugs from the MDD that are commonly used by anesthesiologists in the operating room (interchangeably referred to herein as the “OR”) for a particular procedure or other points of care in the facility where drug containers are labeled prior to dispensing to patients. These are usually the injectable drugs. This subset of drugs can optionally be further narrowed into application-specific sets for pediatrics, drug sets stocked in the drug cabinet 29 of the ambulance 1 and used to stabilize patients for transport, etc. . . . .

Although the examples of drugs provided above are controlled substances typically administered in a surgical setting, the present technology can also be used to document and control the inventory of other substances that are not controlled, or non-surgical in nature. The other substances can include any emergency/patient care drugs, regardless of where typically administered. For example, the other substances that are not surgical, or are general patient care in nature can include, but are not limited to Benadryl, Epinephrine, Adrenaline, Naloxone/Narcan, Amyl nitrite, Bronchodilators Alupent (metaproterenol), Brethaire (terbutaline), Maxair (pirbuterol), Metaprel (metaproterenol), Proventil (albuterol), Tornalate (bitolterol), Ventolin (albuterol), Xopenex (levalbuterol) . . . and the like.

Once the pharmacist selects the drugs for the formulary and assigns the associated information to each drug, a formulary “package” is created. This package is a single electronic file containing all formulary information in a format suitable for delivery to the computer terminals 10 on which the formulary is to be stored. Assembling the formulary into a single package simplifies the transfer of information from the terminal operating the AT to the intended computer terminals 10. It also ensures that all information for that version of the formulary to be transferred to the computer terminals 10 is encapsulated in a single file so no information is lost or forgotten. The formulary package is then transmitted over the network 40 to the computer terminals 10 intended to receive the formulary package, as selected using the AT. According to alternate embodiments, the formulary package can optionally be stored on a USB flash drive and delivered to the computer terminals 10 by plugging the USB flash drive into the computer terminals 10 that are to receive the formulary package, which is then automatically installed. This makes the transfer an all-or-nothing proposition, meaning that the existing formulary on the computer terminals 10 is completely replaced by the formulary package being transferred. If the received formulary package is incomplete or corrupt, it will not be able to be installed on the computer terminals 10, and the user will be alerted to the installation failure.

In addition to delivering formulary packages, the computer terminals 10 accept other types of packages for configuration and software updates. Any of these packages can be delivered via USB drive or network. All packages are encoded with a digital signature to prevent the contents of the package being altered or corrupted. Additionally, the USB flash drive can optionally be required to possess a predetermined digital signature to ensure that only authorized USB flash drives can be plugged into the computer terminals 10 to install a formulary, configuration or software update package.

For example, a configuration package 32 stored in the memory of the computer terminals 10 controls the behavior of those computer terminals 10 when preparing and labeling syringes. It can be used to enable or disable features of the computer terminals 10 such as requiring verification that the drug information displayed on touch-screen display 14 matches the drug container scanned by scanner 18 before printing the label. A pharmacist, head of anesthesia or other authorized individual can customize the workflow to dictate how syringe preparation will be handled and use the configuration package to cause the computer terminals 10 to conform to that desired. Once the configuration package is installed, the computer terminals 10 can impose that workflow on the user (e.g., requiring an authorized confirmation). Multiple workflows can be installed on any given computer terminal 10. In some cases, a user can be granted permission to select a workflow for their use on computer terminal 10. A workflow can optionally be selected based on a user's login information. This allows different workflows for different users. For example, a new resident in the anesthesia program may have all extra verification enabled while a senior physician may have a different workflow configuration. Each workflow can define a sequence of actions to be performed, and optionally is required to be performed, by a user when interacting with the computer terminals 10.

From time to time the software such as the operating system on the computer terminals 10 may need to be updated and/or upgraded. A software update package from a proprietor of the computer terminals 10 may be created and transmitted on a USB flash drive, CD, and/or over a communication network to a hospital for installation on the computer terminals 10, which may change or improve the operation of the system.

Each formulary, configuration and software update package has an identifier string and version number. The identifier can provide human readable information that describes the contents of the package (e.g. Pediatric formulary). A unique version number is assigned to formularies and configuration packages automatically by the AT or from the vendor in the case of software update packages. The combination of the identifier string and version number makes each package easy to identify and track. The computer terminals 10 can display this information on the touch-screen display 14 or send it over the network 40 for remote monitoring. This is useful for tracking which systems have been updated and which system have not.

As described above, a plurality of different formularies may be needed for different purposes. One formulary may contain drugs for general adult surgeries while another may contain different drugs or preparations (dilutions) for pediatric procedures. The AT allows multiple formularies to be created and managed from a single MDD. The user interface of the AT that controls the deployment of formulary packages over the network 40 allows the user to select a single computer terminal 10, as might be required for testing a new version of a formulary before wide-scale deployment, or a plurality or all of the computer terminals 10. In the case of multiple computer terminals 10, these can be manually selected or pre-assigned in groups so all computer terminals 10 in a group can receive the same formulary.

Related to the installation of packages such as formularies, a distribution list of authorized computer terminals 10 can optionally be encoded with the formulary package or other packages such as the configuration package or software update package. The distribution list defines which computer terminals 10 are allowed to install the package. A computer terminal 10 checks the distribution list before installing the package to see if it is on the list. If the computer terminal 10 is not on the distribution list, the package will not be installed. In other words, rather than individually selecting the computer terminals 10 using the AT to which the package is to be transmitted, the computer terminals 10 that are intended to receive each package can be included in the distribution list in the packages themselves. The packages can then be transmitted via the network to all computer terminals 10, but installed on only those computer terminals 10 included on the distribution list. This is particularly useful when a facility uses USB flash drives to distribute packages, but can also apply to network installed packages. For example, a USB flash drive containing a formulary package for general adult surgery might be inadvertently be plugged into a computer terminal 10 intended for pediatric use. The distribution list embedded in the package prevents the pediatric computer terminal 10 from installing the formulary package for the general adult surgery onto the computer terminal 10 intended for pediatric use.

Each computer terminal 10 can optionally be limited to store a single formulary at a time, but alternate embodiments can allow a plurality of different formularies to be installed and selected by the user as the user logs into the computer terminal 10. Alternately, a formulary could be tied to, and automatically selected as the active formulary based on the login information of the user when that user logs in. This would allow a Gastroenterologist, for example, to recognize a different set of drugs with the computer terminals 10 for minor procedures than an anesthesiologist for general surgeries.

In another embodiment, a single formulary 36 can contain drug information suitable for multiple types of procedures such as pediatric, cardiac, general surgery, gastroenterology, minimally invasive surgery and others in a single formulary. The user of computer terminal 10 can select the type of procedure being performed. The type of procedure selected would correspond to a specific subset of drugs and associated drug information contained in formulary 36. For example, a specific drug may not require dilution when used in typical adult surgeries, but may require dilution in pediatric procedures. A single formulary can have different information for preparing the same drug based on the type of procedure currently selected. Additionally, configuration data 32 can be used to limit the procedure types available to a particular user. For example, an anesthesiologist may have full access to all procedures, but a gastroenterologist may be limited to drugs suitable for procedures such as colonoscopies.

Related to a single formulary containing drug information for multiple types of procedures, a default selection of the procedure type can be made based on the user login information on computer terminal 10.

When packages are deployed to the computer terminals 10 over the network, options can be specified that determine when the packages will be installed. It is undesirable to cause a package to be installed in the middle of a medical procedure, so options to defer package installation until the user logs out of the computer terminal 10, or after a specific time, such as 10 PM, or a combination of options such as the first time no user is logged in to the computer terminal 10 and the time is after 10 PM. Other options such as install on next reboot are also possibilities. An optional time delay can be specified that will not immediately install a package when a user logs out. This is to handle the case where one physician goes on break during a long procedure and another physician fills in for the physician on break. In this case, a logout may be followed by another login because the procedure is still underway. A reasonable delay is needed to ensure another user is not going to login. This can also be accomplished by displaying a warning message on the touch-screen display 14 that a package is about to install and a delay to allow the user to touch the screen to defer the installation, providing enough time and notification for the user to log into the computer terminal 10.

Each computer terminal 10 is designed to operate autonomously. Once it has a formulary and configuration package installed, the computer terminals 10 will operate with or without a network connection. Usage data, patient data, configuration data and other information can be stored locally, in the memory module 24 of the computer terminal 10, for example, until a time when the locally-stored information can be transmitted to the healthcare destination and/or dispatch center, for example, as described herein. This ensures the device will continue to work and not interfere with the medical procedure even if the network connection stops functioning. While the network is not functioning the computer terminals 10 will store information that needs to be transmitted for logging, record keeping, billing, and other purposes when the network connection is re-established.

When the computer terminals 10 are connected to the network 40 and the network connection is functioning properly, they can perform other functions in addition to receiving packages. For example, the computer terminals 10 can transmit information regarding the status of the: hardware (e.g., the printer 26 is low or out of a particular printing ink or toner, the printer is out of label stock), package information such as versions of packages installed, the user logged into each of the computer terminals 10 (if any), important events such as “drug not found” alert in response to scanning a barcode with the scanner 18, for example, which may indicate a drug is in the hospital that was not included in the formulary on that computer terminal 10 and may not be properly usable, etc. In such situations, an alert signal is transmitted by the afflicted computer terminal(s) 10 to the email server 46, and the email server 46 responds by composing the email or other message containing textual information corresponding to the alert signal and transmitting the email or other message to the intended recipient. The status information can optionally be transmitted by the computer terminals 10 automatically, not in response to receiving a status request, upon the occurrence of an event, periodically, when a status changes, or a combination thereof. According to an alternate embodiment, the AT running on the terminal 42 or 44 can be used to access the computer terminals 10 over the network 40 to determine the status of each computer terminal 10, the various components making up the computer terminals 10, or other information regarding the computer terminal 10. Thus, the AT running on the terminal 42 or 44 can be used to receive status report information autonomously transmitted by the computer terminals 10, and/or can be used to retrieve (or request transmittal of) the status report information from the computer terminals 10. The status report information can optionally be tabulated by the AT running on the terminal 42 or 44 and presented in a logical manner to the user, thereby allowing the user to readily identify any of the computer terminals 10 that are not operating as intended.

In another embodiment, event information that occurs on a computer terminal 10 can be shared with other computer terminals 10 on the network 40 either through the AT running on one or more of the terminals 42, 44, or the email server 46, or with a dedicated software program on the network 42, or directly with other computer terminals 10 on the network 42. Shared information can be used to optimize the workflow of the users by sharing events such as first-time verification of a drug being used at a computer terminal 10 so other users will have the benefit of the drug verification and not have to perform the same verification procedure on each computer terminal 10.

Related to the aforementioned sharing of information between computer terminals 10 on the network 40, a syringe or other container labeled by the computer terminal 10 can include a unique identifier in a machine-readable format on the label. For example, a unique serial number could be assigned to each syringe and encoded in a barcode that is applied to the syringe. Information related to the unique identifier numbers of the containers prepared at a particular computer terminal 10 and information about the drug in the container (e.g., drug name, concentration, expiration date and/or time, other information, and any combination thereof) can be shared with other computer terminals 10 on the network 40 so a container that is prepared for one patient but is moved to another operating room can be verified when the machine-readable code is scanned by the scanner of the computer terminal 10 in the other operating room. As a result of scanning the barcode or other machine-readable code, a notification can be provided to the user, alerting the user that the drug within that drug container is not intended for that patient (i.e., it is intended for the patient in the original operating room). Alternately, for drug containers permitted to be moved between operating rooms, the contents of the container can be verified in each operating room, and whether the contents are expired, by scanning the machine-readable code with the scanner 18 provided in each of those operating rooms.

Messages of importance to users such planned updates to software, formularies, configuration changes or even messages such as staff meetings or departmental messages can be sent out over the network 40 from an AT running on terminals 42, 44 to one or more computer terminal 10 systems on the network and displayed on the touch-screen display 14 when the user logs into the system. If the message is received on a computer terminal 10 while a user is logged in, a non-intrusive message will notify the user that a message is waiting to be displayed. This will prevent any interruption of the user in the middle of a medical procedure. Messages can be configured to display once per user or each time a user logs in until the message is discontinued from the terminal(s) 42, 44 running the AT. Authority to send out or discontinue messages can optionally be granted or restricted to specific users of the AT.

The usefulness and effectiveness of computer terminal 10 can be enhanced by associating patient information with a medical procedure. Patient information at a healthcare facility is usually stored on an Electronic Medical Record (EMR) system. The EMR typically collects and manages patient Personal Health Records (PHR) from sources throughout the healthcare facility and makes those records available to authorized users and equipment through the network. As related to computer terminals 10, patient information can be transmitted over the network 40 to one or more of the computer terminals 10 from an EMR system in the healthcare facility using HL7 or another healthcare specific network protocol. Patient information such as patient name, ID, date of birth, sex, medical conditions, drug history and other relevant information from the EMR is received and stored by an EMR gateway server. The EMR gateway server can collect and aggregate patient information received from the EMR when the EMR transmits information over the network 40. In other words, the EMR gateway server receives information such as ADT (admission-discharge-transfer) codes and other HL7 messages transmitted from the EMR to different devices intended for different recipients over the network 40. Each such transmission from a plurality of different EMR servers can optionally be collected and recorded by one common gateway server or a plurality of gateway servers. Thus, the information collected by the EMR gateway server can be accessed and retrieved from the EMR gateway server rather than from the EMR server. Since patient information is often transmitted on the network from the EMR as it becomes available from different sources in the healthcare facility, it is necessary to collect and combine the patient information so the appropriate information it is available for a specific purpose. The EMR gateway server performs this function for the computer terminals 10. The EMR gateway server can also reduce the cost of connectivity to the EMR because many EMR systems have a fee per connection and it can be less expensive to connect one EMR gateway server to the EMR than many individual computer terminal 10 systems. An EMR, an EMR in combination with an EMR gateway server, and a plurality of EMR systems in network communication with a common EMR gateway server are represented generally at 47 in FIG. 3. Patient information is transmitted from the EMR gateway server 47 on network 40 to computer terminals 10 when a specific patient identity is entered into a computer terminal 10 or requested from a computer terminal 10 before the patient that is to receive medical attention at a location, such as in an operating room of a healthcare facility, in a pharmacy, or other desired location, for example, where the computer terminal 10 is located. The specific patient's identity can be selected from a patient list stored by the EMR gateway server 47 and accessed using the computer terminal 10, or determined in response to the user entering unique patient identification information such as a patient ID using touch-screen display 14 or scanner 18, for example. The patient ID or other patient-related information can be transmitted over the network and used to look up the patient information from the EMR gateway server. The patient information received from the EMR gateway server by computer terminal 10 is verified by the user using touch-screen display 14 and stored in memory 24 for the duration of the procedure. Likewise, the user can enter, or select from a list displayed via the display 14, the specific procedure to be performed, which is stored in the memory 24 and associated with the patient information. The procedure can optionally also be transmitted from the computer terminal 10 over the network 40 to be stored in association with an entry for that patient in the EMR gateway.

Patient information related to drug allergies, other drugs the patient is taking and relevant information such as date of birth, sex, weight, etc. can affect the selection of medications and doses administered during a medical procedure. Patient information can be associated with a procedure on computer terminal 10 as described above. In the simplest use case, the patient information locally stored in memory 24 on the computer terminal 10 can be displayed on touch-screen display 14 for review by the user. In more complex implementations, the patient information in memory 24 can be accessed by the processing component 22 of the computer terminal 10 and checked as drugs are being prepared on computer terminal 10 to provide warnings to the user if a drug(s) being prepared and labeled is not suitable, or is not apparently suitable to be administered to the patient based on the patient information available. Based on the patient information, information in the formulary, the procedure identified by the user, or any combination thereof, other analyses can be performed, such as verification that the formulary or type of procedure selected as described above or gleaned from the content of a formulary tailored for a particular patient/surgical procedure is appropriate for this patient, patient drug allergies, drug to drug interactions, age related medication restrictions, etc. While performing such an analysis on the computer terminal 10 is one option, a more sophisticated analysis may be possible by communicating with a server included as part of the network 40 that receives individual requests for drug verification along with an indication for selecting the appropriate patient information from the computer terminal 10 and transmits a response to the computer terminal 10 that approves the use of the drug or provides the user with an appropriate warning that is displayed on touch-screen display 14.

Patient information associated with a procedure on computer terminal 10 as described above, can be used to provide drug tracking information for billing and patient records. As drugs are being prepared on computer terminal 10, the drug related information can be transmitted along with information required to associate the drug information with a patient to the EMR gateway server 47. The EMR gateway server 47 then transmits the drug related information along with associated patient information to the EMR 47 at the facility over network 40 using HL7 or another healthcare specific network protocol compatible with the EMR 47.

In another embodiment, the Patient information associated with a procedure on computer terminal 10 as described above, can be used to transmit information to a LIS (Laboratory Information System) 97 in the facility, shown in FIG. 3. The LIS 97 includes a network connected storage device such as a database server for example, that stores records of laboratory samples that are to be, or have been, subjected to medical testing at the laboratory in a computer-readable medium. In many surgical procedures, it is common to have a specimen removed from the patient that is sent to the laboratory for analysis. The specimen is often labeled by hand with patient information, tissue information, site information, date and time of extraction, attending physician, etc., and sent to the laboratory. The computer terminal 10 can allow the user to print a label with the same information that would normally be written by hand and transmit an electronic record of the data to the LIS 97 for storage, so the information on the label of the specimen will exactly match the information stored by the LIS 97 when the specimen arrives in the laboratory.

The computer terminals 10 can transmit data over the network to one or more of the terminals 42, 44 running the AT, a network-connected server, or other network resource, for example, that can be used to generate and analyze drug usage patterns based on procedure type, user or other relevant parameters. As drugs are being prepared by the user using a computer terminal 10, information about the drug including the drug name, concentration, container ID, date, time, user and procedure information can be stored in memory 24 on computer terminal 10 and then transmitted to the terminal(s) running the AT or a dedicated server. The information can then be post processed to extract the required information for determining usage patterns of drugs.

AIMS (Anesthesia Information Management Systems), also known as ARKS (Anesthesia Record Keeping Systems), includes a server, represented generally at 77 in FIG. 3, that receives and stores drug usage information for each patient during a medical procedure to allow anesthesiologists to electronically record patient vital signs, drugs administered, important events that occurred during the surgery and other relevant information related to anesthesia administration and monitoring during a procedure. Many AIMS systems are programmed with a set of all drugs that could be administered in the operating room. This can be hundreds of drugs. When recording a drug in the AIMS 77, the user of the AIMS 77 usually navigates through multiple levels of menus to find the correct drug. A more efficient and accurate method of drug selection can be implemented using network 40 and computer terminal 10 to transfer drug information as they are prepared to the AIMS 77. The AIMS 77 user would then be presented with a short list of the drugs prepared on the computer terminal 10 when they record drug information on the AIMS 77. If the drug is not found on the short list of drugs prepared on computer terminal 10, then the user would have the option to access the full list of drugs stored on the AIMS 77. Although the email server 46, EMR 47, AIMS 77 and LIS 97 appear in FIG. 3 as separate, distributed computational platforms, it is to be understood that one or more of such platforms can be combined and embodied on a single computational platform without departing from the scope of the present invention.

Computer terminal 10 can optionally include a speaker 17 that plays audio files in response to the scanning of a barcode on a drug container by the scanner 18 during preparation of a label. Computer terminal 10 stores audio files or files that can be used to create audible sounds in memory 24. These audio files are executed by the computer terminal 10 to “speak” a drug name and concentration from the speaker 17 when a user scans a drug container using scanner 18. This provides audible confirmation to the user of the drug that was scanned. Other devices on the network that want to provide audible output of drug names, concentrations values and concentration units can transmit a message to computer terminal 10 over the network using a defined interface and message format to instruct computer terminal 10 to audibly “speak” the specified drug name and concentration information. The message can optionally include volume information. Alternately, the other device can transmit a message to computer terminal 10 using a defined interface and message format to select and receive the sound files from the computer terminal 10 and play the sound files locally on the device.

In another embodiment, the computer terminal 10 can transmit a list of prepared drug information over the network 40 to an administration device (e.g., an infusion pump, IV drip, etc.) that is mounted near the point of drug administration to the patient. The administration device (not shown) can include a scanner similar to scanner 18 provided to the computer terminal 10, a display device for displaying the results of scanning a barcode or other machine-readable code, a processing component for converting a scanned code to the identity of the content of the container labeled with the barcode, and a network adaptor to receive the list of prepared drug information over the network. Optionally, the administration terminal can also include a speaker to audibly output the information pertaining to the content of the container labeled with the barcode when the barcode is scanned. The display device and/or the speaker can also optionally output a warning about the container and/or the drug therein in response to reading the barcode and determining that a warning is warranted.

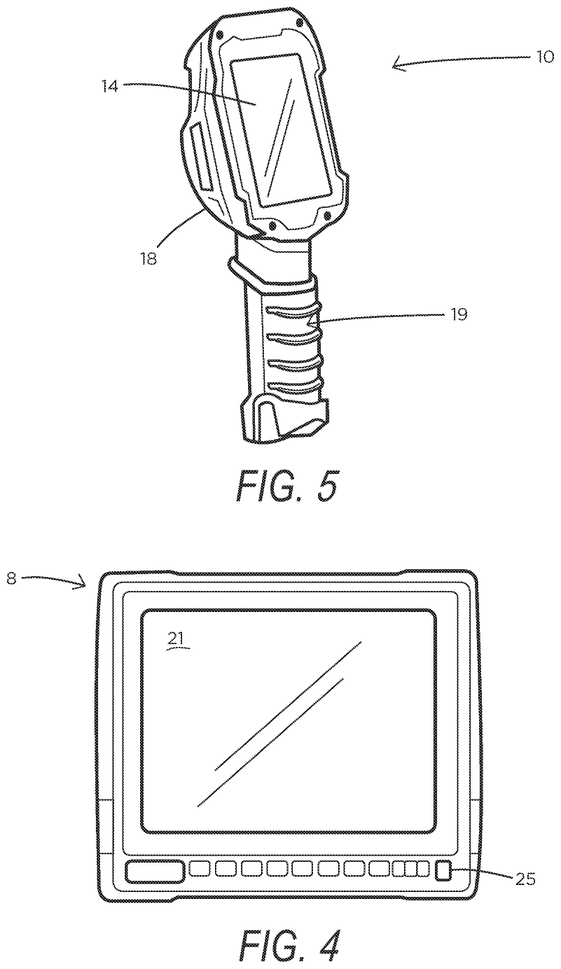

Although the computer terminal 10 is described above as a stationary fixture that can be installed at a location within a hospital or other healthcare facility where drugs are dispensed and administered to patients, alternate embodiments of the computer terminal 10 can be installed in the patient module 4. According to alternate embodiments, the computer terminal 10 can be configured as a handheld device as depicted in FIG. 5, or configured to be integrated into a wall panel, cabinet, or other fixture within the patient module 4 as shown in FIG. 4. For example, a mounting system 11 can engage a portion of the computer terminal 10, such as a handle 19 of the handheld embodiment as shown in FIG. 8, to couple the computer terminal 10 to the ambulance 1. The mounting system 11 can include a clasp, ring or other releasable fastener that establishes a mechanical, friction fit to support the handheld computer terminal 10, yet allow the handheld computer terminal 10 to be readily grabbed and removed to be transported by hands throughout the patient module 4 and put into use as described herein. According to other embodiments, the mounting system can form a permanent coupling of the computer terminal 10 to the ambulance 1. For example, the permanent coupling can optionally interfere with the release of the computer terminal 10 from the ambulance without the use of one or more tools and without damaging the mounting system 11. Such embodiments securely support at least the processing component 22 of the computing system 10 at a fixed location within the mobile medical care environment.

As shown in FIG. 4, the terminal 10 can be integrated into, and configured as part of the communication terminal 8 to be mounted in a dashboard, wall 9 (FIG. 8) or other housing that forms a portion of the ambulance 1 or patient module 4, or other mobile environment for transporting medical patients. As shown, a display device 21 allows a user to enter drug-identifying information to specify the drug to be consumed and administered to a patient. The terminal can utilize realtime/online communication systems, optionally integrated with LIFEPAK 15 emergency care and data connectivity to easily and securely collect and send patient information is needed. Drug access and use information, for example, can be transmitted to clinical information systems, optionally along with documentation of drug and diluent used and tied to patient ID so the data can be communicated before or after the patient arrives at the healthcare-related destination, or immediately upon arrival. Compliance with the National Pre-Hospital and Hospital Data Integration (The National EMS Information System (NEMSIS) as a national standardize for the type of data collected by EMS agencies allows collecting, storing, and sharing standardized EMS data from states nationwide.

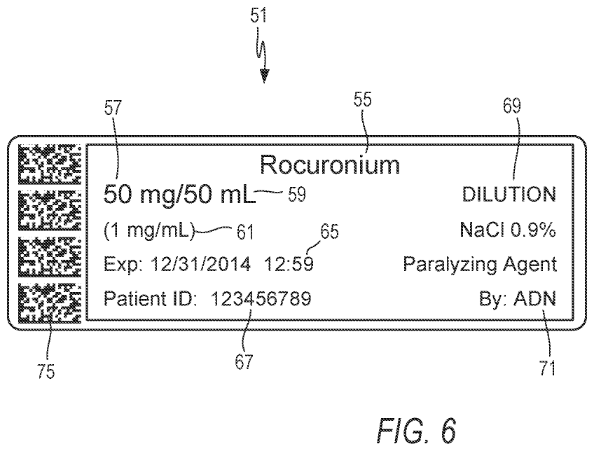

According to another embodiment shown in FIG. 5, a first hand-held terminal 10 includes an integrated scanner 18 built into the handheld terminal 10. Rather than have an EMT or other operator manually enter drug information via a touch-sensitive display 21, the scanner 18 can scan a barcode, RFID tag, or other computer-readable code provided to a drug vial for example. The terminal 10 can interpret the computer-readable code into the associated drug, and document usage to maintain stock information within the mobile environment. For example, the terminal 10 can reference an “approved” UDI/NDC drug database and maintain “on-hand” stock-levels, and optionally transmit a command to print a label 51 such as that shown in FIG. 6, for example, to a printer onboard the ambulance 1 (e.g., within the patient module 4). The terminal 10 allows for fast scanning of drug with audible/visual confirmation of drug in hand based on a computer-readable code such as the National Drug Cod (“NDC”). When not in use, the handheld terminal 10 can be coupled to a wall 9 (FIG. 8) of the patient module 4 by a bracket 11 that acts as a base securing the handheld terminal to the wall 9, from where the handheld terminal 10 can be readily accessed for use as described herein.

The illustrative example of the label 51 shown in FIG. 6 includes a combination of: (i) data determined in response to scanning the computer-readable code provided to the drug vial using the scanner 18, and (ii) data input from at least one other, different source. Examples of the other, different source of data include, but are not limited to: a computer-readable code such as a barcode on a badge worn by, or otherwise associated with the clinician; a computer-readable code such as a barcode on a wristband or other object worn by, or associated with the patient; the clinician who manually inputs data such as the clinician's username or other form of identification; etc.

According to the specific example shown in FIG. 6, at least one of the drug name 55, drug concentration 61, an expiration date and/or time 65 at which the syringe should not be used to administer a drug, and a classification of the drug (e.g., “Paralyzing Agent” in FIG. 6) as stored by the vial can be determined by the handheld terminal 10 in response to scanning a barcode or other computer-readable code associated with a vial containing the drug. If the drug has been diluted from its concentration stored in the vial (the concentration identified in response to scanning the barcode associated with the vial), that information can be manually input by the clinician into the handheld terminal 10. For example, the identifier 69 indicating that the drug is a dilution, and/or a diluent (e.g., saline solution “NaCl 0.9%” in FIG. 6) can be input to the handheld terminal 10 or other embodiment of the computer terminal 10 through keystrokes using a keyboard 25 (FIG. 4) or softkeys for example, by scanning a computer-readable code such as a barcode associated with a container of the diluent, by selecting the diluent from a menu displayed by the computer terminal 10, etc. According to some embodiments, a total dose 57, total volume 59 of the diluted substance to be contained by the syringe can also be calculated by the computer terminal 10 based on a volume of the diluent to be added, or otherwise input to the computer terminal 10.

Also shown in FIG. 6, the embodiment of the label 51 also includes the clinician's initials 71 or other identifying information that can be used to identify a person who is responsible for preparation of the syringe and/or content of the label 51. Such information can be determined by scanning a barcode or other computer-readable code on a badge worn by the clinician or otherwise associated with the clinician and/or the ambulance 1. According to alternate embodiments, the clinician whose initials 71 or other identifier is to appear on the label 51 can be determined by the computer terminal 10 based on login credentials entered by the clinician upon accessing the computer terminal, based on service records (e.g., the name or badge number of personnel on duty with the ambulance 1 at a time when the syringe is to be prepared) for the ambulance 1, etc.

Likewise, the patient identifier 67 can include a name, or other information such as a serial number that does not personally identify the patient. Such information can be manually assigned and/or input by the clinician assigned to the ambulance 1 using the keyboard 25, softkeys, etc. According to other embodiments, the patient can be given a wristband or other badge that contains a barcode or other computer-readable code, which can be scanned by the scanner 18 to identify the patient, and create the label 51.

When the information input to the computer terminal 10 is complete, the label 51 can be generated to include barcode 75 or other computer-readable code that can subsequently be scanned to identify at lease a portion (but less than all) of the content appearing on the label 51, optionally all of the content appearing on the label 51, or optionally all or a portion of the content appearing on the label 51 in combination with other information that does not appear on the label.

According to some embodiments, the label 51 can be generated as a virtual label 16 displayed by the display 14. According to other embodiments, the clinician responsible for preparing the syringe can optionally input a command to the terminal 10, instructing the terminal 10 to transmit a signal causing a printer 26 to publish a hardcopy of the label 51 with an adhesive backing to be applied to the syringe. According to alternate embodiments, the terminal 10 can optionally be configured to generate a record linking the label content 34, or a portion (optionally less than all) of the label content 34 to an electronic record in a database for a patient being transported by the ambulance 1. Such a record can be generated in addition to printing the hardcopy of the label 51, or instead of (i.e., without) printing the hardcopy of the label 51. According to some embodiments, such a record can be generated in addition to generating and displaying the virtual label 16, or instead of (i.e., without) generating and/or displaying the virtual label 16.

According to another embodiment shown in FIG. 4, the terminal integrated as part of the communication terminal 8 includes a keyboard 25, and can optionally also include an integrated scanner 18 such as that built into the handheld terminal 10 shown in FIG. 5. Such an embodiment allows an EMT or other operator to manually and/or automatically enter drug information via the keyboard 25 or the optional scanner 18 by scanning a barcode, RFID tag, or other computer-readable code provided to a drug vial for example. The terminal 10 can interpret the computer-readable code into the associated drug, and document usage to maintain stock information within the mobile environment. For example, the terminal 10 can reference an “approved” UDI/NDC drug database and maintain “on-hand” stock-levels. The terminal 10 allows for fast scanning of drug with audible/visual confirmation of drug in hand based on a computer-readable code such as the National Drug Cod (“NDC”).

Any of the embodiments of the terminal 10 can utilize realtime/online communication systems, optionally integrated with LIFEPAK 15 emergency care and data connectivity to easily and securely collect and send patient information as needed. Drug access and use information, for example, can be transmitted to clinical information systems, optionally along with documentation of drug and diluent used and tied to patient ID so the data can be communicated before or after the patient arrives at the healthcare-related destination, or immediately upon arrival.

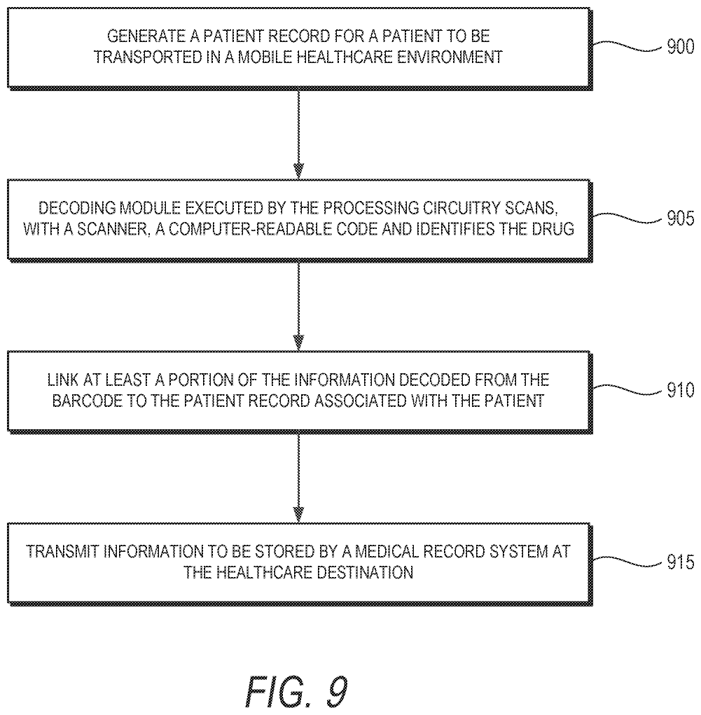

A flow diagram graphically depicting an illustrative method of documenting the use and/or administration of a drug is shown in FIG. 9. A clinician may elect to administer a drug to a patient within the patient module 4, at a location geographically remote from the healthcare destination. The clinician can optionally input patient-specific information such as a name, patient ID number, or any other data uniquely identifying the patient at the time of transport into the computer terminal. At block 900 of FIG. 9, processing circuitry of the computer terminal 10 can generate a patient record specific to the patient.

To document consumption and/or administration of the drug, the clinician can optionally access a drug vial containing the drug to be administered from the drug cabinet 29 in the patient module 4. According to some embodiments, the clinician may remove and carry the handheld embodiment of the computer terminal 10 from the mounting system 11 and scan a barcode or other computer-readable code on the drug vial using the scanner 18 of the handheld computer terminal 10. According to other embodiments, a built-in or peripheral scanner 18 of the permanently-installed embodiment of the computer terminal 10 can scan the computer-readable code. At block 905, a decoding module stored by the memory 24 and executed by the processing component 22 scans the barcode and decodes the barcode symbology to identify the drug.

At block 910, a record module stored by the memory 24 includes instructions that, when executed by the processing circuitry 22, causes the processing circuitry 22 to link at least a portion of the information decoded from the barcode on the drug vial to the patient record that is associated with the patient and stored in the memory 24. For example, at least one of the drug name, NDC number (or other drug identification number), lot number, concentration of the drug to be administered, identification of the clinician, and any other information that can appear as label content 34 (FIG. 1) on a label such as that shown in FIG. 6 can be linked to the patient record.

At block 915, a communication module stored in the memory 24 and executed by the processing component 22 can include instructions that, when executed by the processing circuitry component 22, cause the processing component 22 to transmit information to be stored by a medical record system at the healthcare destination. For example, the communication module can modulate and transmit at least one of: (i) the portion of the decoded information from the barcode on the drug vial that is linked to the patient ID, and (ii) the patient ID to be stored by the medical record system (e.g., electronic medical record “EMR”) at the healthcare destination. Transmission of such information can optionally occur before the ambulance 1 arrives at the geographic location of the healthcare destination over a wide area network. For example, before the ambulance with the patient departs for a hospital or other healthcare destination, or as the ambulance is underway, traveling en route to the healthcare destination, the data transmission can be facilitated over a wide area network such as a cellular network, satellite communication network, or terrestrial network.

According to alternate embodiments, the transmission of such information can be delayed until the ambulance 1 arrives at the healthcare destination, or otherwise comes within the communication range of a local area network associated with the healthcare destination. For example, the patient ID and the information related to the drug can be stored locally in the memory 24 until a time when the ambulance 1 is within range of the local area network. Upon arriving at the healthcare destination, a dispatch station associated with the ambulance 1, or any other destination location where the ambulance 1 is within range of the local area network, the communication module can transmit the information and upload the data to an EMR associated with the same patient at that destination location.

Illustrative embodiments have been described, hereinabove. It will be apparent to those skilled in the art that the above devices and methods may incorporate changes and modifications without departing from the general scope of this invention. It is intended to include all such modifications and alterations within the scope of the present invention. Furthermore, to the extent that the term “includes” is used in either the detailed description or the claims, such term is intended to be inclusive in a manner similar to the term “comprising” as “comprising” is interpreted when employed as a transitional word in a claim.

1. A computing system within a transport vehicle constituting a mobile medical care environment, the transport vehicle comprising a drug dispensary storing a supply of medicinal substances available for use in administering care to a patient within the transport vehicle, the computing system comprising:
a mounting system comprising a base that is coupled to a structure of the transport vehicle and supports the computing system within the transport vehicle;
processing circuitry;
a non-contact scanner;
a memory coupled to the processing circuitry and storing computer executable instructions that, when executed by the processing circuitry, cause the processing circuitry to:
(i) scan, with the non-contact scanner, a computer-readable code encoding information about a first medicinal substance stored by the drug dispensary, and (ii) determine the portion of the encoded information based on a signal transmitted by the non-contact scanner in response to scanning the computer-readable code, by looking up an entry corresponding to content transmitted by the signal in a formulary locally stored in non-transitory computer-readable medium provided to the transport vehicle;
link at least a portion of the encoded information to a patient ID that is associated with the patient and stored in the memory; and
transmit: (i) the portion of the encoded information linked to the patient ID, and (ii) the patient ID to be stored by a medical record system at a destination medical care environment, wherein the encoded information and the patient ID are stored locally in the non-transitory computer-readable medium provided to the transport vehicle while unable to communicate with the medical record system at the destination medical care environment, and are transmitted to the medical record system after the transport vehicle is located at the destination medical care environment and able to communicate with the medical record system.

2. The computing system of claim 1, wherein the base is coupled to the structure of the transport vehicle by a mechanical fastener, securely supporting at least the processing circuitry of the computing system at a fixed location within the transport vehicle.

3. The computing system of claim 1, wherein the base is coupled to the structure of the transport vehicle by a mechanical fastener, and releasably supports at least the non-contact scanner so the non-contact scanner can be transported and used at different regions within the transport vehicle.

4. The computing system of claim 3, wherein the base further releasably supports the processing circuitry so the non-contact scanner and the processing circuitry can be transported and used as a handheld apparatus at the different regions within the transport vehicle.

5. The computing system of claim 1, wherein the non-contact scanner comprises at least one of a RFID reader and a barcode reader that scans a compatible computer-readable code applied to a label on a container storing the first medicinal substance.

6. (canceled)

7. The computing system of claim 1, wherein the content transmitted by the signal includes a National Drug Code, and the portion of the encoded information is looked up from the entry including the National Drug Code in the formulary.

8. The computing system of claim 1, wherein the portion of the encoded information comprises at least a name of the medicinal substance, and is determined from the formulary locally within the transport vehicle by the computing system without communicating with a remote computing terminal located externally of the transport vehicle.

9. The computing system of claim 1 further comprising, a local area network antenna comprising a communication range that is: (i) insufficient to communicate with the medical record system at the destination medical care environment while the transport vehicle is traveling along a portion of a route to the destination medical care environment, and (ii) sufficient to communicate with the medical record system when the transport vehicle is located at the destination medical care environment.

10. (canceled)

11. The computing system of claim 1 further comprising, an interface with a communication system provided to the transport vehicle to transmit the portion of the encoded information to the medical record system substantially over the communication system of the transport vehicle.

12. A method of documenting a drug within a transport vehicle, the transport vehicle comprising a drug dispensary storing a supply of medicinal substances available for use in administering care to a patient within the transport vehicle, the method comprising:
with a computing system mounted within the transport vehicle, scanning a computer-readable code encoding information about a first medicinal substance stored by the drug dispensary with a non-contact scanner;
linking at least a portion of the encoded information to a patient ID that is associated with the patient in the transport vehicle and stored in the memory; and
transmitting via an antenna provided to the transport vehicle: (i) the portion of the encoded information linked to the patient ID, and (ii) the patient ID to be stored by a medical record system at a destination medical care environment wherein the portion of the encoded information and the patient ID are: (i) locally in the memory provided to the transport vehicle and not transmitted to the medical record system while the transport vehicle is out of a communication range of the medical record system at the destination medical care environment, and (ii) transmitted to the medical record system after the transport vehicle is located at the destination medical care environment and within the communication range of the medical record system.

13. The method of claim 12 further comprising, determining the portion of the encoded information based on a signal transmitted by the non-contact scanner in response to scanning the computer-readable code, by looking up an entry corresponding to content transmitted by the signal in a formulary locally stored in a non-transitory computer-readable medium.

14. The method of claim 13, wherein the content transmitted by the signal includes a National Drug Code, and the portion of the encoded information is looked up from the entry including the National Drug Code in the formulary.

15. The method of claim 13, wherein the portion of the encoded information comprises at least a name of the medicinal substance, and is determined from the formulary locally within the transport vehicle by the computing system without communicating with a remote computing terminal located externally of the transport vehicle.
